(19) United States
(12) Patent Application Publication
(10) Pub. No.: US 2008/0233076 A1
(43) Pub. Date: Sep. 25, 2008

(54) Process for stabilizing ascorbic acid compositions

(75) Inventors: Isabelle Hansenne, Westfield, NJ (US); Hani Fares, Somerset, NJ (US); Dana Smith, Monmouth Jct, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/726,319

(22) Filed: Mar. 21, 2007

(51) Int. Cl. A61K 31/745

(52) U.S. Cl. 424/78.03

(57) ABSTRACT

The present invention relates to a process for inhibiting radical-induced damage to skin involving contacting the skin with a composition containing: a) ascorbic acid and/or a water soluble derivative thereof; b) at least one cinnamic acid derivative; c) water; d) at least one non-aqueous organic solvent; e) at least one pH adjusting agent; and, f) optionally, a surfactant, and wherein the pH of the composition is from about 4 to about 9.

### BACKGROUND OF THE INVENTION

Aging skin is the result of more than just chronological age. Skin is exposed to environmental elements that cause radicals to form in the skin. These radicals attack the collagen layer of the skin and break it down, causing lines and wrinkles to appear. This process is commonly called photo-aging. Diseases and disorders of skin that also may result from radical damage include skin cancer, skin irritation or inflammation, dermatitis, allergy, psoriasis, acne, eczema, rosacea.

Application of antioxidants can help reduce the effects of radical-induced damage in skin. Applying Vitamin C, for example, to the skin can provide antioxidant protection, prevent photo-aging, and stimulate collagen production. However, not all Vitamin C formulations produce these benefits due to lack of stability of the vitamin in the formulation.

Several approaches have been proposed for the stabilization of ascorbic acid in formulations. These include low pH (U.S. Pat. No. 5,140,043 to Darr, D. and Pinnell, S.) wherein the composition has a pH no greater than 3.5, or the addition of cinnamic acid derivatives such as ferulic acid to a composition comprising ascorbic acid, also at a pH no greater than about 3.5 (US Patent application No. US20050154054).

Unstable solutions of ascorbic acid develop a yellow, or even brown discoloration, and result in carbon dioxide gas formation. This is due to the decarboxylation of the molecule within the package, which can present dangers of explosion if kept closed for long periods of times.

It has been surprisingly discovered that compositions comprising ascorbic acid and cinnamic acid derivatives, wherein the compositions have a pH value of from about 4 to about 9, are efficacious as topical compositions and, at higher pH, do not suffer from conventional stability issues.

### BRIEF SUMMARY OF THE INVENTION

A first aspect of the present invention is directed to a process for inhibiting radical-induced damage to skin involving contacting the skin with a composition containing: a)ascorbic acid and/or a water soluble derivative thereof; b) at least one cinnamic acid derivative; c) water; d) at least one non-aqueous polar organic solvent; e) at least one pH adjusting agent; and, optionally, f) a surfactant, and wherein the pH of the composition is from about 4 to about 9.

A second aspect of the present invention relates to a composition containing: a) ascorbic acid and/or a water soluble derivative thereof; b) at least one cinnamic acid derivative; c) water; d) at least one non-aqueous polar organic solvent; e) at least one pH adjusting agent; and, optionally, f) a surfactant, and wherein the pH of the composition is from about 4 to about 9.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

According to one embodiment of the present invention, there is provided an ascorbic acid-containing composition which is both efficacious at inhibiting radical-induced damage in skin and which, at higher pH, does not suffer from conventional stability issues.

Examples of “radical-induced damage” include, but are not limited to, photo-aging; diseases/disorders of the skin such as skin cancer, skin irritation/inflammation, dermatitis, allergy, psoriasis, acne, eczema and rosacea.

Ascorbic Acid and Derivatives

Ascorbic acid in aqueous solutions is readily degraded into oxidized forms that subsequently become a source of free radicals. The oxidation reactions consume ascorbic acid and reflect on the stability of ascorbic acid. The degradation of ascorbic acid is also accompanied by the development of a brown color and the formation of carbon dioxide.

Ascorbic acid is commercially available from Sigma-Aldrich (St. Louis, Mo.), for example. In one embodiment, the L-ascorbic acid is purchased at 99% purity.

Ascorbic acid derivatives also contemplated in the invention include those derivatives which exhibit water solubility. By soluble, it is meant it exhibits a solubility of at least 0.1% by weight, such as at least 1% by weight, such as at least 5% by weight, in water at 25° C. In other words, it does not form a distinct separate phase (liquid or solid) under the conditions specified. Suitable ascorbic acid derivatives include, but are not limited to ascorbyl glucoside and magnesium ascorbyl phosphate.

The ascorbic acid and water soluble derivatives thereof are present in the cosmetic composition in an amount ranging from greater than about 1 to about 40% by weight, such as from about 5 to about 30% by weight, such as from about 10 to about 20% by weight, all weights based on the total weight of the composition.

Cinnamic Acid Derivatives

Cinnamic acid derivatives which improve the stability of ascorbic acid are contemplated to be included in the compositions of the present invention.

Suitable cinnamic acid derivatives include, but are not limited to, caffeic acid, p-coumaric acid, ferulic acid, sinapinic acid, combinations thereof, cis and trans isomers thereof, salts thereof, and equivalent derivatives thereof.

Cinnamic acid derivatives are present in the compositions of the present invention in an amount of from about 0.2 to about 5.0% by weight, such as from about 0.5 to about 4% by weight, such as from about 0.5 to about 2% by weight, all weights based on the total weight of the composition.

Non-Aqueous Polar Organic Solvent

A variety of non-aqueous polar organic solvents may be used in the composition of the present invention. Examples thereof are as follows.

Polyols

Polyols are suitable nonaqueous polar organic solvents. For purposes of this specification, polyols are defined as compounds which contain three or more hydroxyl groups per molecule. Examples of suitable polyols include fructose, glucamine, glucose, glucose glutamate, glucuronic acid, glycerin, 1,2,6-hexanetriol, hydroxystearyl methylglucanine, inositol, lactose, maltitol, mannitol, methyl gluceth-10, methyl gluceth-20, methyl glucose dioleate, methyl glucose sesquicaprylate/sesquicaprate, methyl glucose sesquicocoate, methyl glucose sesquiisostearate, methyl glucose sesquilaurate, methyl glucose sesquistearate, phytantriol, riboflavin, sorbeth-6, sorbeth-20, sorbeth-30, sorbeth-40, sorbitol, sucrose, thioglycerin, xylitol, and mix thereof. An especially preferred polyol is glycerin.

Polymeric or Monomeric Ethers

Also suitable as the non-aqueous polar organic solvent are homopolymeric or block copolymeric liquid ethers. Polymeric ethers are preferably formed by polymerization of monomeric alkylene oxides, generally ethylene or propylene oxides. Examples of such polymeric ethers include PEG, PPG, and derivatives thereof.

Other examples of suitable polymeric ethers include polyoxypropylene polyoxyethylene block copolymers. Such compounds are sold under the CTFA name Meroxapol 105, 108, 171, 172, 174, 178, 251, 252, 254, 255, 258, 311, 312, and 314.

Mono- and Dihydric Alcohols

Also suitable for use as to the non-aqueous polar organic solvent are mono- and dihydric alcohols of the general formula R(OH)ₙ where n is 1 or 2 and R is a substituted or unsubstituted saturated C₂₋₁₀, preferably C₁₋₈ alkyl, or a substituted or unsubstituted alicyclic, bicyclic, or aromatic ring, with the substituents selected from halogen, alkoxy, hydroxy, and so on. Examples of suitable alcohols include monohydric alcohols such as ethanol, isopropanol, hexyldecanol, benzyl alcohol, propyl alcohol, and isopropyl alcohol, as well as dihydric alcohols such as hexylene glycol, diethylene glycol, ethylene glycol, propylene glycol, 1,2-butylene glycol, 1,5-pentanediol, triethylene glycol, dipropylene glycol, and mixtures thereof.

Sorbitan Derivatives

Sorbitan derivatives, which are defined as ethers or esters of sorbitan, are also suitable polar solvents. Examples of suitable sorbitan derivatives are the Polysorbates, which are defined as stearate esters of sorbitol and sorbitan anhydrides, such as Polysorbate 20, 21, 40, 60, 61, 65, 80, 81, and 85. Also suitable are fatty esters of hexitol anhydrides derived from sorbitol, such as sorbitan trioleate, sorbitan tristearate, sorbitan sesquistearate, sorbitan stearate, sorbitan palmitate, sorbitan oleate, and mixtures thereof.

In one embodiment, glycol ethers such as di(ethylene glycol) ethyl ether, also known as ethoxy diglycol, 2-(2-ethoxyethoxy)ethanol, diglycolmonoethyl ether, ethyl diethylene glycol, ethylene diglycol monoethyl ether, CARBITOL®, or TRANSCUTOL®, may be used. Di(ethylene glycol) ethyl ether is commercially available from Sigma-Aldrich. Other glycol ethers include methoxyisopropanol, PPG-2 methyl ether, PPG-3 methyl ether, propylene glycol butyl ether, PPG-2 butyl ether, phenoxyisopropanol, butoxyethanol, butoxydiglycol, methoxydiglycol, phenoxyethanol, PPG-3 butyl ether, PPG-2 propyl ether, propylene glycol propyl ether, or dipropylene glycol dimethyl ether, for example, from the Dow Chemical Company, Midland, Mich.

In some embodiments, the ratio of glycol ether to alkanediol may be about 1.5:1, 2:1, 3:1 and up to about 4:1. In particular, the ratio of glycol ether to alkanediol may be about 2:1.

The non-aqueous polar organic solvent may be present in the composition of the present invention in an amount of up to about 60% by weight, such as up to about 15%, up to about 20%, up to about 25%, up to about 30%, up to about 35%, up to about 40%, up to about 45%, up to about 50%, or up to about 55% by weight, all weights based on the total weight of the composition.

pH Adjusting Agents

pH adjusting agents are used to adjust the pH of the composition to a value of from about 4 to about 9, such as from about 5 to about 8, such as from about 5 to about 7; such as to about 6. Suitable pH adjusting agents include, but are not limited to, sodium hydroxide, potassium hydroxide, ammonium hydroxide, sodium carbonate, potassium carbonate, amino methyl propanol, potassium phosphate, dipotassium phosphate, sodium citrate, potassium citrate, and mixtures thereof.

Suitable pH adjusting agents may also include, but not be limited to, buffers such as citrate buffers, or phosphate buffers which will allow the composition to resist changes in pH.

Surfactant

A surfactant may be needed in the composition of the present invention in the event the composition contains hydrophobic agents such as Vitamin E, or concentrations of a cinnamic acid derivative at greater than 0.5% by weight, for example, to facilitate solubilization. Surfactants may also be needed in the event the composition of the invention is an emulsion.

The term “surfactant” is defined, in accordance with the invention, as a compound having at least one hydrophilic moiety and at least one lipophilic moiety. The surfactants may be organic surfactants or silicone surfactants or mixtures thereof.

Suitable organic surfactants include, but are not limited to, nonionic surfactants, such as polyoxyethylene sorbitan monolaurate (TWEEN®), i.e., TWEEN®20), polyoxyethylene 23 lauryl ether (BRIJ®-35) or polyoxyethylated octyl phenol (TRITON®); zwitterionic surfactants such as 3-((3-cholamidopropyl) dimethylammonio)-1-propane sulfonate (CHAPS®) or coco dimethyl carboxymethyl betaine; cationic surfactants such as behentrimonium chloride; or an anionic surfactant such as cholate, deoxycholate, sodium dodecylsulfate, or TWEEN®-80; and mixtures thereof.

Also suitable as surfactants may be silicone surfactants.

Suitable silicone surfactants that may used in the compositions of the present invention may be liquid or solid at room temperature and are generally a water-in-oil or oil-in-water type surfactants which are preferably nonionic.

The silicone surfactant used in the compositions of the invention is an organosiloxane polymer containing a polymeric backbone including repeating siloxy units that may have cyclic, linear or branched repeating units, e.g. di(lower)alkylsiloxy units, preferably dimethylsiloxy units. The hydrophilic portion of the organosiloxane is generally achieved by substitution, onto the polymeric backbone, of a radical that confers hydrophilic properties to a portion of the molecule, either on the end-unit of the polymeric organosiloxane, or on any one or more repeating units of the polymer. It should also be understood that the organosiloxane polymer in accordance with the invention should have at least one hydrophilic portion and one lipophilic portion.

The term “hydrophilic radical” means a radical that, when substituted onto the organosiloxane polymer backbone, confers hydrophilic properties to the substituted portion of the polymer. Examples of radicals that will confer hydrophilicity include, but are not limited to, hydroxy-polyethyleneoxy, hydroxyl, carboxylates, sulfonates, sulfates, phosphates, and amines.

The term “lipophilic radical” means an organic radical that, when substituted onto the organosiloxane polymer backbone, confers lipophilic properties to the substituted portion of the polymer. Examples of organic radicals which will confer lipophilicity are C₁₋₄₀ straight or branched chain alkyl, fluoro, aryl, aryloxy, C₁₋₄₀ hydrocarbyl acyl, hydroxypolypropyleneoxy, or mixtures thereof. The C₁₋₄₀ alkyl may be non-interrupted, or interrupted by one or more oxygen atoms, a benzene ring, amides, esters, or other functional groups.

Suitable examples of silicone surfactants that may be used in the compositions of the invention include, but are not limited to, those sold by Goldschmidt under the ABIL™ trademark including ABIL B-9806™; those sold by Rhone-Poulenc under the Alkasil™ tradename; those sold by Amerchol under the Amersil™ tradename, including Amersil ME-358™, Amersil DMC-287™ and Amersil DMC-357™; those sold by Dow Corning as Dow Corning 3225C™ Formulation Aid, Dow Corning 190™ Surfactant, Dow Corning 193™ Surfactant, Dow Corning Q2-5200™; those sold by Union Carbide under the tradename Silwet™; those sold by Troy Corporation under the Troysol™ tradename; those sold by Taiwan Surfactant Co. under the tradename Ablusoft™; those sold by Hoechst under the tradename Arkophob™; and mixtures thereof.

Additional compounds suitable as surfactants include, but are not limited to, emulsifying crosslinked siloxane elastomers such as Dimethicone/PEG-10/15 Crosspolymer available as KSG-210, Dimethicone/Polyglycerin-3 Crosspolymer available as KSG 710, Lauryl PEG-15 Dimethicone/Vinyl Dimethicone Crosspolymer available as KSG-31, PEG-12 Dimethicone Crosspolymer, available as DC 9011. In one embodiment, the compound useful as a surfactant is Dimethicone/PEG-10/15 Crosspolymer.

The composition of the present invention may contain at least one surfactant in an amount of from about 0.01 to about 15% by weight, such as from about 0.5 to about 10% by weight, such as from about 1 to about 8% by weight, all weights based on the total weight of the composition.

Auxiliary Ingredients

Vitamin E

The compositions may also further comprise Vitamin E. Vitamin E is available as alpha, beta, delta, and gamma tocopherols, alpha, beta, delta and gamma tocotrienols, and mixtures thereof and derivatives thereof. In one embodiment, the form of Vitamin E is an alpha, beta, delta, or gamma tocopherol and, in another embodiment, the form of Vitamin E is an alpha tocopherol. Salts or derivatives of tocopherols include pharmaceutically acceptable compounds such as acetate, sulfate, succinate, nicotinate, palmitate, allophanate, phosphate, quinone, or halogenated derivatives, esters, or stereoisomers, for example. The invention encompasses the use of Vitamin E derivatives in which substitutions, additions, and other alterations have been made in the 6-chromanol ring and/or side chain, with the proviso that the derivatives maintain the antioxidant activity of Vitamin E. Additional tocopherols can be constructed by conjugation to the ring structure or side chain of various other moieties, such as those containing oxygen, nitrogen, sulfur and/or phosphorus. Tocopherol derivatives can also be made by modifying the length of the side chain from that found in tocopherols such as alpha-, beta-, delta- and gamma-tocopherol. Tocopherols can also vary in stereochemistry and saturation of bonds in the ring structure and side chain.

Additional tocopherol derivatives, including prodrugs, can be made by conjugation of sugars or other moieties to the side chain or ring structure. Tocopherols include without limitation stereoisomers (e.g., + and − stereoisomers of alpha-tocopherol; (+/−) indicates a racemic mixture) or mixtures of structurally distinct tocopherols (e.g., alpha- plus gamma-tocopherol). Tocopherols may be obtained from Roche, Nutley, N.J., for example.

Additional Solvents

Additional cosmetically acceptable solvents may be used in the compositions of the present invention. Suitable for use as additional solvents are non-aqueous non-polar organic solvents.

A variety of non-aqueous non-polar organic solvents can be used in the composition of the invention, if so desired. Examples thereof are as follows.

Silicones

Silicones are suitable non-polar compounds. The silicones may be volatile or non-volatile. The term “volatile” means that the silicone has a measurable vapor pressure, i.e. a vapor pressure of at least 2 mm. of mercury at 20° C. If volatile, the silicone generally will have a viscosity of 0.5 to 25 centistokes at 25° C. Suitable volatile silicones include cyclic silicones, linear silicones, or mixtures thereof.

Linear and cyclic volatile silicones are available from various commercial sources including Dow Corning Corporation and General Electric. These fluids comprise octamethylcyclotetrasiloxane, decamethylcyclopentasiloxane, hexamethyldisiloxane, and mixtures thereof which are available from Dow Corning, for example, and sold under the tradenames Dow Corning 244, 245, 344, and 200 fluids.

The silicones may also be nonvolatile, and in particular water insoluble nonvolatile silicones. The term “nonvolatile” means that the silicone has a vapor pressure of less than 2 mm. of mercury at 20° C. A variety of silicones fits this definition including dimethicone, phenyl trimethicone, diphenyl dimethicone, methicone, hexadecyl methicone, stearoxydimethicone, stearyl dimethicone, cetyl dimethicone, and so on.

Cyclomethicone is a preferred silicone for use in the composition of the invention.

Esters

In addition to the sorbitan esters, other esters are also suitable as the non-aqueous non-polar organic solvent. In general such esters have the formula R¹CO—OR² wherein R¹ and R² are independently a C₁₋₂₅ straight or branched chain saturated or unsaturated alkyl, alkylcarbonyloxyalkyl, or alkoxycarbonylalkyl, aryl, which may be substituted or unsubstituted with halogen, hydroxyl, alkyl, and the like.

Suitable esters may include, but are not limited to, alkyl acetates, alkyl behenates, alkyl lactates, alkyl benzoates, alkyl octanoates, alkyl salicylates, and in particular C₁₂₋₁₅ alkyl benzoate, and mixtures thereof. Examples of further esters are set forth on pages 502-506 of the CTFA Cosmetic Ingredient Handbook, Second Edition, 1992, which is hereby incorporated by reference.

Fats and Oils

Fats and oils are also suitable as the non-aqueous non-polar organic solvent. Preferably these materials are liquids or semi-solids at room temperature. They are generally defined as glyceryl esters of fatty acids (triglycerides), as well as the synthetically prepared esters of glycerin and fatty acids. Suitable fats and oils may include, but are not limited to, oils such as apricot kernel oil, avocado oil, canola oil, olive oil, sesame oil, peanut oil, soybean oil, trilinolenin, trilinolein, trioctanoin, tristearin, triolein, sesame oil, rapeseed oil, sunflower seed oil, and mixtures thereof.

Fatty Acids

Fatty acids are also suitable as the non-aqueous non-polar organic solvent in the compositions of the invention. Preferably the fatty acids are liquid or semi solid at room temperature. Fatty acids are the carboxylic acids obtained by hydrolysis of animal or vegetable fats and oils. Carboxylic acids having alkyl chains shorter than about seven carbon atoms are not generally considered fatty acids. Fatty acids have the general structure R³—COOH where R³ is a straight or branched chain saturated or unsaturated C₇₋₆₅ alkyl. Suitable fatty acids may include, but are not limited to, arachidic acid, arachidonic acid, behenic acid, capric acid, caproic acid, caprylic acid, coconut acid, corn acid, cottonseed acid, hydrogenated coconut acid, hydroxystearic acid, lauric acid, linoleic acid, linolenic acid, linseed acid, myristic acid, oleic acid, palmitic acid, palm kernel acid, soy acid, tallow acid, and mixtures thereof.

Fatty Alcohols

Fatty alcohols may also be used as the non-aqueous non-polar organic solvent. Fatty alcohols are generally made by reducing the fatty acid —COOH group to the hydroxyl function. They generally have the formula R⁴CH₂OH. Suitable fatty alcohols may include, but are not limited to, behenyl alcohol, C₉₋₁₁ alcohol, C₁₂₋₁₃ alcohol, C₁₂₋₁₅ alcohol, C₁₂₋₁₆ alcohol, caprylic alcohol, cetearyl alcohol, cetyl alcohol, coconut alcohol, decyl alcohol, lauryl alcohol, myristyl alcohol, oleyl alcohol, and mixtures thereof.

Hydrocarbons

Hydrocarbons are also good non-aqueous non-polar organic solvents in accordance with the invention. Suitable hydrocarbons may include, but are not limited to, C₇₋₆₀ isoparaffins, ethane, heptane, hexane, hydrogenated polyisobutene, isobutane, isododecane, isoeicosane, isohexadecane, isopentane, microcrystalline wax, mineral oil, mineral spirits, paraffin, petrolatum, petroleum distillates, squalene, polyethylene, and mixtures thereof. Preferred hydrocarbons are mineral oil and polyethylene.

Lanolin and Lanolin Derivatives

Also suitable as the non-aqueous non-polar organic solvent are lanolin and derivatives thereof. Suitable lanolin and lanolin derivatives may include, but are not limited to, acetylated hydrogenated lanolin, acetylated lanolin alcohol, laneth, lanolin acid, lanolin oil, lanolin alcohol, lanolin wax, and mixtures thereof.

The additional solvent may be present in the composition of the present invention in an amount of up to about 60% by weight, such as up to about 15%, up to about 20%, up to about 25%, up to about 30%, up to about 35%, up to about 40%, up to about 45%, up to about 50%, or up to about 55% by weight, all weights based on the total weight of the composition.

Sunscreens

The composition of the present invention may also contain sunscreens. A sunscreen is defined as an ingredient that absorbs at least 85 percent of the light in the UV range at wavelengths from 290 to 320 nanometers, but transmit UV light at wavelengths longer than 320 nanometers. Sunscreens generally work in one of two ways. Particulate materials, such as zinc oxide or titanium dioxide, in microfine form, absorb ultraviolet radiation. Chemical sunscreens, on the other hand, operate by chemically reacting upon exposure to UV radiation. Suitable sunscreens that may be included in the compositions of the invention are set forth on page 582 of the CTFA Cosmetic Ingredient Handbook, Second Edition, 1992, as well as U.S. Pat. No. 5,620,965, both of which are hereby incorporated by reference. Suitable sunscreen materials may include, but are not limited to, p-aminobenzoic acid (PABA), cinoxate, diethanolamine p-methoxycinnamate (DEA-methoxycinnamate), Digalloyl trioleate, dioxybenzone (Benzophenone-8), ethyl 4-[bis-(hydroxypropyl)] aminobenzoate (ethyl dihydroxypropyl PABA), 2-ethylhexyl-2-cyano-3,3-diphenylacrylate (octocrylene), ethylhexyl p-methoxycinnamate (Octyl methoxycinnamate), 2-ethylhexyl salicylate (Octyl salicylate), glyceryl aminobenzoate (Glyceryl PABA), homosalate, lawsone with dihydroxyacetone, menthyl anthranilate, oxybenzone (Benzophenone-3), Padimate A (Pentyl Dimethyl PABA), Padimate 0, (octyl Dimethyl PABA), 2-Phenylbenzimidazole-5-sulfonic acid (Phenylbenzimidazole Sulfonic acid), Red Petrolatum, Sulisobenzone (Benzophenone-4), triethanolamine salicylate (TEA-Salicylates), and mixtures thereof.

The sunscreens may be present in an amount ranging from about 0.001 to about 20% by weight, such as from about 0.01 to about 10% by weight, such as from about 0.05 to about 8% by weight, all weights based on the total weight of the composition.

Preservatives

Preservatives having antibacterial activity are optionally present in the compositions of the present invention. Representative examples of preservatives include, but are not limited to, alkyl para-hydroxybenzoates, wherein the alkyl radical has from 1, 2, 3, 4, 5 or 6 carbon atoms and preferably from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Mixtures of preservatives may certainly be used, e.g., the mixture of methyl-paraben, ethylparaben, propylparaben and butylparaben sold under the name Nipastat by Nipa, and the mixture of phenoxyethanol, methylparaben, ethylparaben, propylparaben and butylparaben sold under the name Phenonip, also by Nipa. Also suitable are 4-hydroxy benzoic acid, benzoic acid, sorbic acid, dehydroacetic acid, triclosan, benzyl alcohol, chlorophenesin, and salicylic acid.

Preservatives may be present in amounts ranging from about 0.01 to about 10% by weight, preferably from 0.5 to about 5% by weight, and more preferably from about 0.8 to about 3% by weight, all weights based on the total weight of the composition.

Moisturizers

Moisturizers are optionally present in the compositions of the present invention. Any moisturizer commonly used in cosmetic formulations is an acceptable moisturizer for the compositions herein, such as Panthenol (pro-Vitamin B5), commercially available from Sigma-Aldrich. Panthenol has additional desirable biological properties, such as wound healing properties.

Viscosity Enhancers

A viscosity enhancer is optionally present in the compositions of the present invention. Any viscosity enhancer commonly used in cosmetics is acceptable for compositions herein. Sodium hyaluronate is an example of a viscosity enhancer that also provides a slip effect that improves the feeling of the composition on the skin. Sodium hyaluronate also assists in keeping moisture on the skin and improves absorption of the composition. Carboxymethylcellulose, for example, is another viscosity enhancer commonly used in cosmetics.

Additional auxiliary ingredients include, for example, cosmetic or dermatological ingredients known to one of skill in the art, including further antioxidants such as Vitamin A derivatives such as a retinoid, retinol, retinal, retinoic acid, a retinoic acid salt, a derivative or analog thereof, or a mixture thereof, lipoic acid, seleno-L-methionine, or flavonoids that lack undesirable color. The compositions may also contain mild surfactants, oil components, emulsifiers, pearlizing waxes, consistency factors, thickeners, superfatting agents, stabilizers, polymers, silicone compounds, fats, waxes, lecithins, phospholipids, biogenic agents, further UV protection factors, deodorants, antiperspirants, antidandruff agents, film formers, swelling agents, insect repellents, self-tanning agents, hydrotropes, solubilizers, preservatives, perfume oils, or dyes, for example as further additives.

According to another embodiment of the present invention there is provided a process for inhibiting radical-induced damage to skin involving contacting the skin with the above-disclosed composition.

According to yet another embodiment of the present invention there is provided a process for stabilizing a solution of ascorbic acid and at least one cinnamic acid derivative by raising the pH of said solution to a value of from greater than about pH 5 to about pH 9.

The following examples are presented to further illustrate various aspects of the present invention, and are not intended to limit the scope of the invention.

### EXAMPLES

### Comparative Example 1

### 10% Ascorbic Acid, 0.2% Ferulic Acid at pH 2. (740021 4)

| | Ex. 1 |
|---|---|
| INCI Name | w/w % |
| TOCOPHEROL | — |
| DIPROPYLENE GLYCOL | 20.00 |
| FERULIC ACID | 0.20 |
| PHENOXYETHANOL | 1.00 |
| ASCORBIC ACID | 10.00 |
| GLYCERIN | 3.00 |
| WATER | 62.70 |
| LAURETH-23 | 3.00 |
| SODIUM HYALURONATE | 0.10 |
| Total | 100.00 |

### Comparative Example 2

### 15% Ascorbic Acid, 0.5% Ferulic, 1% Tocopherol at pH 2 (740019 7)

| | Ex. 2 |
|---|---|
| INCI Name | w/w % |
| TOCOPHEROL | 1.00 |
| DIPROPYLENE GLYCOL | 20.00 |
| FERULIC ACID | 0.50 |
| PHENOXYETHANOL | 1.00 |
| ASCORBIC ACID | 15.00 |
| GLYCERIN | 3.00 |
| WATER | 56.40 |
| LAURETH-23 | 3.00 |
| SODIUM HYALURONATE | 0.10 |
| Total | 100.00 |

### Comparative Example 3

### 15.3% Ascorbic Acid, 0.5% Ferulic Acid, 1% Tocopherol (pH 3) (740019 2)

| | Ex. 3 |
|---|---|
| INCI Name | w/w % |
| TOCOPHEROL | 1.00 |
| PROPYLENE GLYCOL | 5.00 |
| FERULIC ACID | 0.50 |
| PHENOXYETHANOL | 1.00 |
| ANTIFOAM | 0.01 |
| ASCORBIC ACID | 15.30 |
| PENTYLENE GLYCOL | 5.00 |
| GLYCERIN | 3.00 |
| SODIUM HYDROXIDE | 0.18 |
| WATER | 56.01 |
| LAURETH-23 | 3.00 |
| SODIUM HYALURONATE (10% solution) | 10.00 |
| Total | 100.00 |

### Inventive Example 4

### 10% Ascorbic, 0.2% Ferulic Acid at pH 6 (740021 5)

| | Ex. 4 |
|---|---|
| INCI Name | w/w % |
| TOCOPHEROL | — |
| DIPROPYLENE GLYCOL | 20.00 |
| FERULIC ACID | 0.20 |
| PHENOXYETHANOL | 1.00 |
| SODIUM HYDROXIDE | 2.24 |
| ASCORBIC ACID | 10.00 |
| GLYCERIN | 3.00 |
| WATER | 60.46 |
| LAURETH-23 | 3.00 |
| SODIUM HYALURONATE | 0.10 |
| Total | 100.00 |

### Inventive example 5

### 15% Ascorbic Acid, 0.5% Ferulic Acid, 1% Tocopherol at pH 6. (740019 6)

| | Ex. 5 |
|---|---|
| INCI Name | w/w % |
| TOCOPHEROL | 1.00 |
| DIPROPYLENE GLYCOL | 20.00 |
| FERULIC ACID | 0.50 |
| PHENOXYETHANOL | 1.00 |
| SODIUM HYDROXIDE | 4.08 |
| ASCORBIC ACID | 15.00 |
| GLYCERIN | 3.00 |
| WATER | 52.32 |
| LAURETH-23 | 3.00 |
| SODIUM HYALURONATE | 0.10 |
| Total | 100.00 |

Control composition without actives ((740028 1)

| | Ex. 6 |
|---|---|
| INCI Name | w/w % |
| DIPROPYLENE GLYCOL | 20.00 |
| PHENOXYETHANOL | 1.00 |
| GLYCERIN | 3.00 |
| WATER | 72.90 |
| LAURETH-23 | 3.00 |
| SODIUM HYALURONATE | 0.10 |
| Total | 100.00 |

In vivo determination of influence of pH on efficacy.

The study was performed on the back of 11 subjects with very fair or fair skins associated to a phototype II. Prior to the first application, each area to be treated was cleansed using an absorbent paper imbibed with water.

All products were applied, twice a day (morning and 4 hours later) for three consecutive days, at a rate of 4 mg/cm², with a finger provided with a finger cot. On day 4, the products were applied only once, and one hour and a half after application, the treated zones were exposed to 320-440 nm UVA at a dose corresponding to 1.5 and 2 Individual Minimal Erythemal Doses (iMED).

Four zones of 4×5 cm² are traced on the back of the subjects: three treated zones, one with comparative composition, example 2 one with comparative composition, example 3, and one with inventive composition example 5; and one with the control composition, example 6, without any actives.

Colorimetric measurements and visual assessments were performed 2 hours after exposure in order to evaluate the intensity of UVA-induced erythema and that of the Persistent Pigment Darkening (PPD).

Colorimetric Measurements

The equipment used for calorimetric measurements was as follows:

Minolta Cr 300 No2 Colorimeter

1600 Watts Xenon lamp ORIEL 4×4 with filters WG 335 3 mm (A) and dichroic mirror for UVA spectrum in the field 320-440 nm, was used for calorimetric measurements in compliance with the in vivo PPD method as described in the following articles: A. Chardon, D. Moyal, C. Hourseau: Persistent pigment darkening response as a method for evaluation of UVA protection assays published in “Sunscreens: development, evaluation and regulatory aspects”, 2nd edition (N. Lowe, N. Shath, M. Pathak, ed.; Marcel Dekker Inc.), pp. 559-582, 1986, and D. Moyal, A. Chardon, N. Kollias: UVA protection efficacy of sunscreens can be determined by the persistent pigment darkening (PPD) method (Part 2), published in Photo-dermatol. Photoimmunol. Photomed., 16, 250-255, 2000.

UV-METER PMA No5 with UVA Sensor SN 2423 used to measure and calculate the amounts of UVA received (J.cm²)

Balance

Finger cot

Evaluation Criteria

Colorimetric measurements were performed on the treated exposed zones (TEZ) and on the non-treated exposed zones (NTEZ) (2 measurements in triplicate). The measurements were expressed as L*, a*, and b* values (CIE 1976 L*a*b* also known as CIELAB). The L*, a*, and b* values obtained from the zones treated with a product were compared to the L*, a*, and b* values obtained from the zones treated with the control formulation.

The UVA-induced erythema is expressed as Δa*, which represents the difference in a* values between the treated exposed zones (TEZ) and the non-treated exposed zones (NTEZ). Δa* represents the red hue, which quantifies the intensity of the UVA-induced erythema. The larger the Δa*, the more intense the erythema.

Visual assessments of the UVA-induced erythema were performed 2 hours after exposure and reported on a scale having 14 levels of evaluation, from 0=No Erythema to 13=darkest pink.

The Persistent Pigment Darkening (PPD) is reported as ΔE, which represents the total modification of the coloring of the skin.

ΔE is expressed as the square root of (Δa*²+Δb*²+ΔL*²).

The larger the ΔE, the more intense the persistent pigment darkening (PPD). ΔE may be used to represent the overall change in color.

Results

1.5iMED Exposure

Colorimetric measurements of UVA-induced erythema after 1.5iMED exposure

Each sample was measured against the control composition Ex. 6, containing only the carrier and no active.

Comparative Example 2 (pH 2), comparative Example 3 (pH 3), and inventive composition Example 5 (pH 6), are not significantly different from each other. Examples 2 and 3 are different from the vehicle (p=0.065 and p=0.082 respectively). Example 5 is not significantly different from the vehicle. The available raw data shows that the erythema caused by 1.5iMED was low and that the variability of the results for Example 5 was greater.

Visual Assessment of UVA Induced Erythema

Each sample was measured against Example 6, which is the control formulation containing only the carrier and no active.

Comparative Example 2 (pH 2), comparative Example 3 (pH 3), and inventive Example 5 (pH 6) are statistically different from the vehicle example 6. There are not significantly different from each other (p>0.05)

Persistent Pigment Darkening (PPD)

Each sample was measured against the control composition Ex. 6, containing only the carrier and no active.

No significant differences exist among the samples and the vehicle.

2iMED Exposure

Colorimetric measurements of UVA-induced erythema after 2iMED exposure

Each sample was measured against the control composition Ex. 6, containing only the carrier and no active.

Comparative Example 2 (pH 2), comparative Example 3 (pH 3) are significantly different from the vehicle, whereas inventive composition example 5 is not significantly different from the vehicle. Comparative Example 2 (pH 2), comparative Example 3 (pH 3), and inventive Example 5 (pH 6) are not significantly different from each other (p>0.5).

Visual Assessment of UVA Induced Erythema at 2MED

Each sample was measured against Example 6, which is the control formulation containing only the carrier and no active.

Comparative Example 2 (pH 2), comparative Example 3 (pH 3), and inventive Example 5 (pH 6) are significantly different from the vehicle. Comparative Example 2 (pH 2), comparative Example 3 (pH 3), and inventive Example 5 (pH 6) are not significantly different form each other (p>0.5).

Persistent Pigment Darkening (PPD) at 2MED

Each sample was measured against the control composition Ex. 6, containing only the carrier and no active.

Comparative Example 2 (pH 2), comparative Example 3 (pH 3) are significantly different from the vehicle, whereas inventive composition Example 5 is not significantly different from the vehicle. Comparative Example 2 (pH 2), comparative Example 3 (pH 3), and inventive Example 5 (pH 6) are not significantly different from each other (p>0.5).

Assessment of Consumer Perceived Differences Due to pH Value.

The study was conducted using a half-face study lasting 4 weeks. Products were blinded and packaged in generic packaging. Subjects were randomly assigned to apply the pH 2 products to one side of the face and the pH 6 products to the other side of the face. Fifty two (52) subjects completed the study. Participants completed a questionnaire containing twelve (12) questions after using the test products for four (4) weeks. The questions were in the form of a nine (9) point semi-structured scale. The scale ranged from one (1) to nine (9), where a score at (1) was the most negative rating, and a score at (9) was the most positive rating the products could receive. Panelists were asked to use comparative composition, example 1 (10% Ascorbic acid, 0.2% Ferulic acid at pH 2) on side A of the face, neck or chest, and inventive composition, example 4 (10% ascorbic, 0.2% ferulic acid at pH 6) on side B of the face, neck or chest for a period of 1 week, followed by the application of comparative composition, example 2 (15% Ascorbic acid, 0.5% ferulic, 1% Tocopherol at pH 2) on side A of the face, neck or chest, and inventive composition, example 5 (15% ascorbic acid, 0.5% ferulic acid, 1% tocopherol at pH 6) on side B of the face, neck or chest for a period of 3 weeks.

Data were tested for normal distribution and the significance level was set at p≦0.05. A Tukey test (using the SAS statistical program) was performed to compare the consumer ratings for each attribute. If statistically significant differences were found, they are reported at the 95% confidence level (p-value≦0.05).

The results are as follows:

| | Mean Score for | Mean Score for | | Statistical |
|---|---|---|---|---|
| | Inventive | Comparative | | Significance |
| Attribute | compositions | compositions | p-value | at p ≦ 0.05 |
| Healthy | 5.38 | 4.98 | 0.0228 | Yes |
| Younger | 5.1 | 4.88 | 0.1395 | No |
| Lifted | 4.87 | 4.46 | 0.0043 | Yes |
| Rejuvenates | 5.31 | 4.96 | 0.0394 | Yes |
| Radiant | 5.35 | 4.85 | 0.0021 | Yes |
| Overall | 5.31 | 5.09 | 0.4014 | No |
| Firmer | 4.87 | 4.73 | 0.4047 | No |
| Smoother | 5.69 | 5.33 | 0.0478 | Yes |
| Softer | 5.77 | 5.41 | 0.0297 | Yes |
| Texture | 5.17 | 4.79 | 0.0037 | Yes |
| Toned | 4.96 | 4.63 | 0.0549 | No |
| Gentle | 6.73 | 6.2 | 0.0016 | Yes |

The analysis of the results demonstrates that the inventive compositions at pH 6 performed at statistical parity for some of the attributes and significantly better than the comparative compositions at pH 2.

Stability of Ascorbic Acid in Compositions Containing Ferulic acid and Vitamin E.

Compositions comprising 15% Ascorbic Acid, 0.5% Ferulic acid and 1% Vitamin E were made according to Example 4, and the pH adjusted to different values (pH 2 to pH 7) with sodium hydroxide. The compositions were stored at 45° C. for 1 month, and the pressure inside the container was measured with a pressure gauge.

Development of pressure vs. pH

| pH | Pressure (psi) |
|---|---|
| 2 | 19.33 |
| 3 | 24.67 |
| 4 | 30.00 |
| 5 | 19.67 |
| 6 | 8.00 |
| 7 | 0.17 |

The results are also reported below in the form of a chart.

The results show the reduction in carbon dioxide at a pH above 4, such as above pH 6, such as above pH6, such as pH 7.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

What is claimed is:

1. A process for inhibiting radical-induced damage to skin involving contacting the skin with a composition comprising:
(a) ascorbic acid and/or a water soluble derivative thereof;
(b) at least one cinnamic acid derivative;
(c) water;
(d) at least one non-aqueous organic solvent;
(e) at least one pH adjusting agent; and,
(f) optionally, a surfactant, and wherein the pH of the composition is from about 4 to about 9.

2. The process according to claim 1, wherein the pH of the composition is from about pH 5 to about pH 7.

3. The process according to claim 1, wherein (a) is present in an amount of from about 1% to about 40% by weight, based on the total weight of the composition.

4. The process according to claim 1, wherein (a) is present in an amount of from about 10% to about 20% by weight, based on the total weight of the composition.

5. The process according to claim 1, wherein (a) is ascorbic acid.

6. The process according to claim 1, wherein (b) is present in an amount of from about 0.2% to about 5.0% by weight, based on the total weight of the composition.

7. The process according to claim 1, wherein (b) is present in an amount of from about 0.5% to about 2% by weight, based on the total weight of the composition.

8. The process according to claim 1, wherein (b) is chosen from p-coumaric acid, ferulic acid, caffeic acid, sinapinic acid, and mixtures thereof.

9. The process according to claim 1, wherein (b) is ferulic acid.

10. The process according to claim 1, wherein (d) is chosen from mono-, di-hydric alcohols, polyols and mixtures thereof.

11. The process according to claim 1, wherein the composition further comprises tocopherol and/or derivatives thereof.

12. A process for stabilizing a solution of ascorbic acid and/or a derivative thereof and at least one cinnamic acid derivative by raising the pH of said solution to a value of from about pH 5 to about pH 9.

13. A cosmetic or dermatological composition comprising:
(a) ascorbic acid and/or a water soluble derivative thereof;
(b) at least one cinnamic acid derivative;
(c) water;
(d) at least one non-aqueous organic solvent;
(e) at least one pH adjusting agent, and;
(f) optionally, a surfactant,
and wherein the pH of the composition is from about 4 to about 9.

14. The composition according to claim 13, wherein the pH of the composition is from about pH 5 to about pH 7.

15. The composition according to claim 13, wherein (a) is present in an amount of from about 1% to about 40% by weight, based on the total weight of the composition.

16. The composition according to claim 13, wherein (a) is present in an amount of from about 10% to about 20% by weight, based on the total weight of the composition.

17. The composition according to claim 13, wherein (a) is ascorbic acid.

18. The composition according to claim 13, wherein (b) is present in an amount of from about 0.2% to about 5.0% by weight, based on the total weight of the composition.

19. The composition according to claim 13, wherein (b) is present in an amount of from about 0.5% to about 2% by weight, based on the total weight of the composition.

20. The composition according to claim 13, wherein (b) is chosen from p-coumaric acid, ferulic acid, caffeic acid, sinapinic acid, and mixtures thereof.

21. The composition according to claim 13, wherein (b) is ferulic acid.

22. The composition according to claim 13, wherein (d) is chosen from mono-, di-hydric alcohols, polyols and mixtures thereof.

23. The composition according to claim 13, wherein the composition further comprises tocopherol and/or a derivative thereof.

24. A cosmetic or dermatological composition comprising:
(a) about 15% by weight ascorbic acid;
(b) about 0.5% by weight ferulic acid;
(c) about 1% by weight tocopherol and/or a derivative thereof;
(d) water;
(e) at least one non-aqueous organic solvent;
(f) at least one pH adjusting agent; and,
(g) a surfactant, all weights based on the total weight of the composition,
and wherein the pH of the composition is from about 5 to about 7.
